(19) United States
(12) Patent Application Publication
(10) Pub. No.: US 2020/0281643 A1
(43) Pub. Date: Sep. 10, 2020

(54) REDUCED CAPACITIVELY LEAKAGE CURRENT IN ELECTROSURGICAL INSTRUMENTS

(71) Applicant: Intuitive Surgical Operations, Inc., Sunnyvale, CA (US)

(72) Inventors: Mathew P. Nussbaum, Campbell, CA (US); Jason W. Hemphill, Los Gatos, CA (US)

(21) Appl. No.: 16/763,977

(22) PCT Filed: Nov. 16, 2018
(86) PCT No.: PCT/US2018/061621

§ 371 Date: May 13, 2020

Related U.S. Application Data
(60) Provisional application No. 62/588,087, filed on Nov. 17, 2017.

(51) Int. Cl. A61B 18/14; A61B 18/12; A61B 34/00

(52) U.S. Cl. CPC A61B 18/1445; A61B 2018/00071; A61B 34/25; A61B 18/1206

(57) ABSTRACT

A surgical instrument includes a shaft that includes an elongated tube having distal and proximal end portions. The tube houses an electrical conductor that includes distal and portions and that extends between the proximal and distal end portions of the elongated tube. An end effector at the distal end portion of the elongated tube includes an electrode electrically coupled to the distal portion of the electrical conductor. A switch at the proximal end portion of the shaft is operatively disposed between the distal and proximal portions of the electrical conductor that is configured to electrically couple and decouple the distal and proximal portions of the electrical conductor.

### CLAIM OF PRIORITY

This application claims the benefit of priority to U.S. Provisional Patent Application Ser. No. 62/588,087, filed on Nov. 17, 2017, which is incorporated by reference herein in its entirety.

### BACKGROUND

Electrosurgery involves the use of electricity to buildup heat within biological tissue to cause thermal tissue damage resulting in incision, removal or sealing of the tissue through one or more of desiccation, coagulation, or vaporization, for example. Benefits include the ability to make precise cuts with limited blood loss. Electrosurgical devices are frequently used during surgical procedures to help prevent blood loss in hospital operating rooms or in outpatient procedures. High-frequency electrosurgery typically involves radio frequency (RF) alternating current (AC) that is converted to heat by resistance as it passes through the tissue.

### SUMMARY

In one aspect, a surgical instrument includes a shaft that includes an elongated tube having distal and proximal end portions. The tube houses an electrical conductor that includes distal and portions and that extends between the proximal and distal end portions of the elongated tube. An end effector at the distal end portion of the elongated tube includes an electrode electrically coupled to the distal portion of the electrical conductor. A switch at the proximal end portion of the shaft is operatively disposed between the distal and proximal portions of the electrical conductor that is configured to electrically couple and decouple the distal and proximal portions of the electrical conductor.

In another aspect, a surgical system includes an electrosurgical instrument including proximal and distal end portions and including an electrode at the distal end portion. An electrosurgical signal generator provides an electrosurgical signal. A first electrical conductor extends between the electrosurgical instrument and the electrosurgical signal generator. A second electrical conductor extends between the proximal and distal end portions of the electrosurgical instrument and is electrically coupled to the electrode. A first switch electrically couples and decouples the electrosurgical signal to and from the second electrical conductor. A second switch to electrically couples and decouples the first and second electrical conductors.

In yet another aspect, a method selectably provides an electrosurgical signal at a surgical instrument end effector. In response to a command at a user interface control to energize an electrosurgical instrument, a first switch is closed to electrically provide an electrosurgical signal to a first electrical conductor and a second switch is closed to electrically couple the first electrical conductor to a second electrical conductor electrically coupled to an end effector. In response to no command at the user interface control to energize the electrosurgical instrument while a hovering gesture is detected at the user interface control, the first switch is opened to not provide the electrosurgical signal to the first electrical conductor and the second switch is closed to electrically couple the first electrical conductor to a second electrical conductor electrically coupled to an end effector. In response to a command at the user interface control to energize the electrosurgical instrument while no hovering gesture is detected at the user interface control, the first switch is opened to not provide the electrosurgical signal to the first electrical conductor and the second switch is opened to electrically decouple the first electrical conductor from the second electrical conductor electrically coupled to an end effector.

### BRIEF DESCRIPTION OF DRAWINGS

FIG. 1 is an illustrative plan view of a minimally invasive surgical system for performing a minimally invasive diagnostic or surgical procedure on a patient who is lying on an operating table.

FIG. 2 is an illustrative perspective view of the surgeon's console.

FIG. 3 is an illustrative perspective view of a patient-side cart of a minimally invasive surgical system.

FIG. 4 is an illustrative perspective view of a surgical instrument.

FIG. 5 is an illustrative schematic diagram showing a first bipolar surgical electrosurgical instrument and second monopolar surgical electrosurgical instrument that may be selectively coupled to receive high frequency signals from an electrosurgical generator unit in accordance with some embodiments.

FIG. 6 is an illustrative partially cut-away, cross-section drawing showing first and second cable isolation switches at the first bipolar electrosurgical instrument in accordance with some embodiments.

FIGS. 7A-7B are illustrative top elevation views of a rotary disk switch embodiment of the first and second cable isolation switches of FIGS. 5-6 in closed and open states, respectively, in accordance with some embodiments.

FIG. 7C is an illustrative cross-section view along line 7C-7C of FIG. 7A showing double wipe electrical switch contacts in accordance with some embodiments.

FIG. 8 is an illustrative perspective view of a motor pack to enclose multiple motors to controllably rotate individual rotary drive disks in accordance with some embodiments.

FIG. 9 is an illustrative drawing representing a lead screw embodiment of a cable isolation switch.

FIG. 10 is an illustrative drawing representing a cam action embodiment of a first cable isolation switch.

FIG. 11 is an illustrative drawing representing user interface controls and a sensor device disposed to sense user proximity in accordance with some embodiments.

### DESCRIPTION OF EMBODIMENTS

### Surgical System

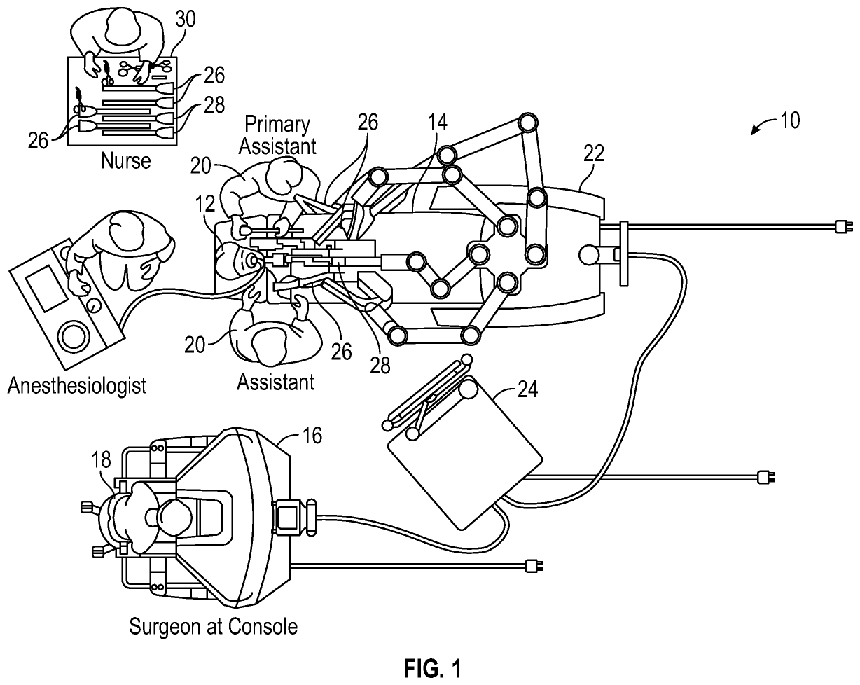

FIG. 1 is an illustrative plan view of a minimally invasive surgical system 10 for performing a minimally invasive diagnostic or surgical procedure on a patient 12 who is lying on an operating table 14. The system includes a surgeon's console 16 for use by a surgeon 18 during the procedure. One or more assistants 20 may also participate in the procedure. The minimally invasive surgical system 10 further includes one or more patient-side cart 22 and an electronics cart 24. The patient-side cart 22 can manipulate at least one surgical instrument 26 through a minimally invasive incision in the body of the patient 12 while the surgeon 18 views the surgical site through the surgeon's console 16. An image of the surgical site can be obtained by an endoscope 28, such as a stereoscopic endoscope, which may be manipulated by the patient-side cart 22 to orient the endoscope 28. Computer processors located on the electronics cart 24 may be used to process the images of the surgical site for subsequent display to the surgeon 18 through the surgeon's console 16. In some embodiments, stereoscopic images may be captured, which allow the perception of depth during a surgical procedure. The number of surgical instruments 26 used at one time will generally depend on the diagnostic or surgical procedure and the space constraints within the operative site among other factors. If it is necessary to change one or more of the surgical instruments 26 being used during a procedure, an assistant 20 may remove the surgical instrument 26 from the patient-side cart 22, and replace it with another surgical instrument 26 from a tray 30 in the operating room.

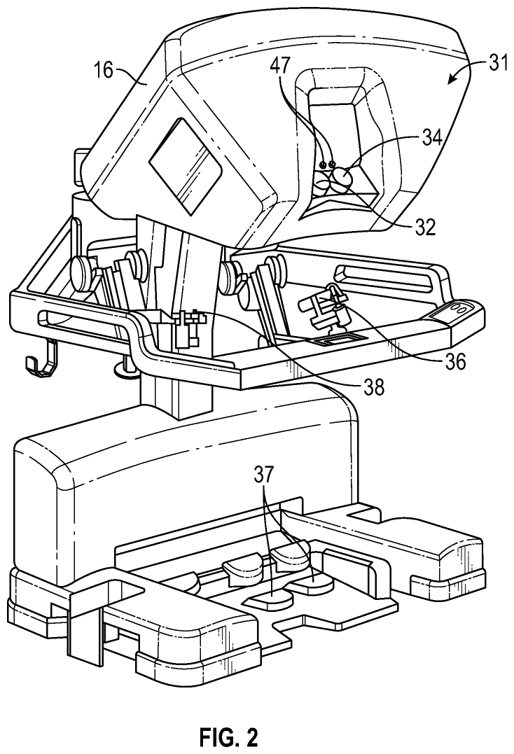

FIG. 2 is a perspective view of the surgeon's console 16. The surgeon's console 16 includes a viewer display 31 that includes a left eye display 32 and a right eye display 34 for presenting the surgeon 18 with a coordinated stereoscopic view of the surgical site that enables depth perception. The console 16 further includes one or more hand-operated user interface control inputs 36 to receive the larger-scale hand control movements and includes one or more foot pedal user interface control inputs 37. The foot pedal user interface control inputs 37 may receive user commands to actuate an electrosurgical instrument. For example, a user may press down upon a foot pedal to indicate a command to energize an electrosurgical instrument, and a user may release a foot pedal to indicate a command to de-energize an electrosurgical instrument. One or more surgical instruments installed for use on the patient-side cart 22 move in smaller-scale distances in response to surgeon 18's larger-scale manipulation of the one or more control inputs 36. The control inputs 36 may provide the same mechanical degrees of freedom as their associated surgical instruments 26 to provide the surgeon 18 with telepresence, e.g., the perception that the control inputs 36 are integral with the instruments 26 so that the surgeon has a strong sense of directly controlling the instruments 26. To this end, position, force, and tactile feedback sensors (not shown) may be employed to transmit position, force, and tactile sensations from the surgical instruments 26 back to the surgeon's hands through the control inputs 36, subject to communication delay constraints.

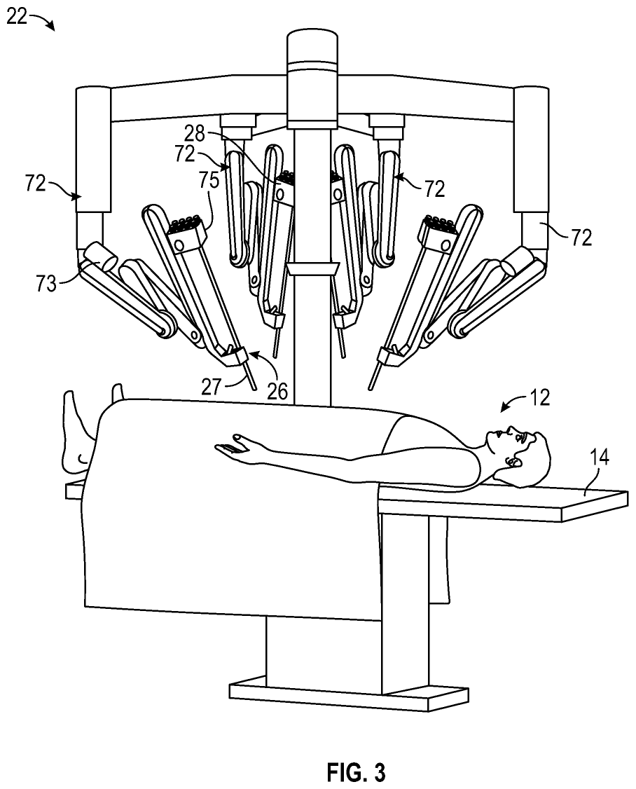

FIG. 3 is a perspective view of a patient-side cart 22 of a minimally invasive surgical system 10, in accordance with embodiments. The patient-side cart 22 includes four mechanical support arms 72. A surgical instrument manipulator 73, which includes motors to control instrument motion, is mounted at the end of each support arm assembly 72. Additionally, each support arm 72 can optionally include one or more setup joints (e.g., unpowered and/or lockable) that are used to position the attached surgical instrument manipulator 73 in relation to the patient for surgery. While the patient-side cart 22 is shown as including four surgical instrument manipulators 73, more or fewer surgical instrument manipulators 73 may be used. A surgical system will generally include a vision system that typically includes an endoscopic camera instrument 28 for capturing video images and one or more video displays for displaying the captured video images. User inputs provided at the control console 16 to control either the instrument as a whole or the instrument's components are such that the input provided by a surgeon or other medical person to the control input (a “master” command) is translated into a corresponding action by the surgical instrument (a “slave” response).

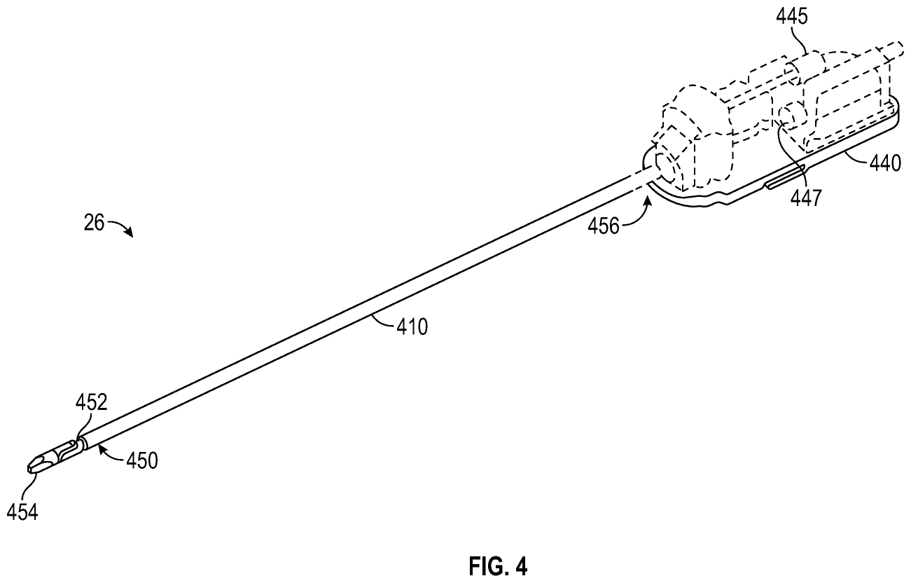

FIG. 4 is a perspective view of a surgical instrument 26, which includes an elongated hollow tubular shaft 410 having a centerline longitudinal axis 411, a distal (first) end portion 450 for insertion into a patient's body cavity and proximal (second) end portion 456 coupled adjacent an end effector actuator mechanism 440 disposed at a proximal end of the instrument 26, which includes multiple mechanical actuators 445, 447 (shown with dashed lines) that may include one or more pulleys, guides, anchors, capstans, levers, linear slides or anchors, for example, cooperatively coupled to exert force upon mechanical wire cables that extend within the shaft 410 and that are coupled to impart motion to the end effector 454 such as opening or closing of jaws and (x, y) wrist motion of a wrist. The surgical instrument 26 is used to carry out surgical or diagnostic procedures. The distal portion 450 of the surgical instrument 26 can provide any of a variety of end effectors 454, such as the forceps shown, a needle driver, a cautery device, a cutting tool, an imaging device (e.g., an endoscope or ultrasound probe), or the like. The surgical end effector 454 can include a functional mechanical degree of freedom, such as jaws that open or close, or a knife that translates along a path or a wrist that may move in x and y directions. In the embodiment shown, the end effector 454 is coupled to the elongated hollow shaft 410 by a wrist 452 that allows the end effector to be oriented relative to the elongate tube centerline axis 411. The end effector actuator 440 controls movement of the end effector at its distal portion.

### Electrosurgical Instruments

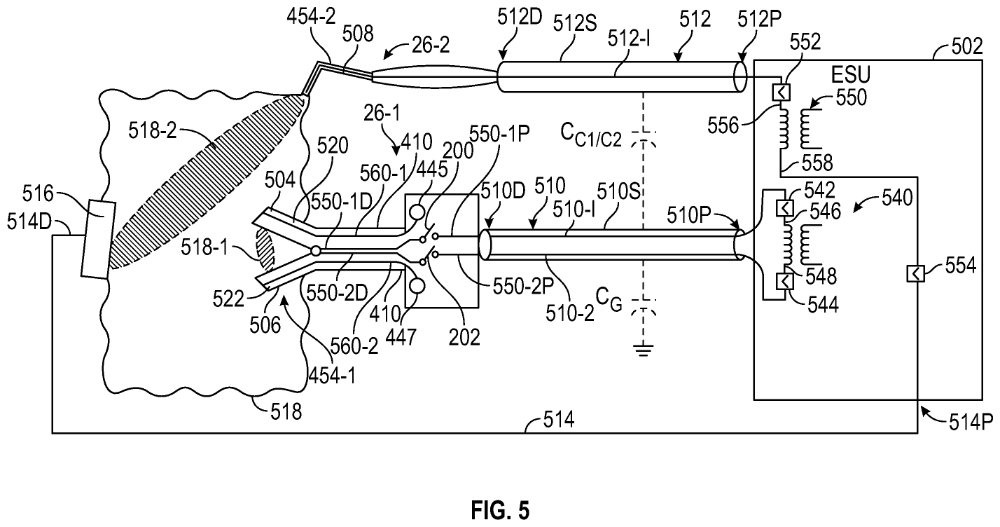

FIG. 5 is an illustrative schematic diagram showing a first bipolar surgical electrosurgical instrument 26-1 and second monopolar surgical electrosurgical instrument 26-2 that may be selectively coupled to receive electrosurgical signals from an electrosurgical generator unit (ESU) 502 in accordance with some embodiments. An electrosurgical signal typically is a high frequency signal (HF) in a radio frequency range (RF) that has a voltage level suitable to achieve a desired surgical effect such as desiccation, coagulation, or vaporization, for example. The voltage level of an electrosurgical signal may be selected according to the desired surgical effect. The first electrosurgical instrument (ESI) 26-1 includes a first end effector 454-1 that includes a first jaw member 520 including a first electrode 504 and a second jaw member 522 that includes a second electrode 506. The second ESI 26-2 includes a second end effector 454-2 that includes a single electrode, a third electrode 508. A first conductor cable 510 extends between the ESU 502 and the first and second electrodes 504, 506 of the first ESI 26-1. A second conductor cable 512 extends between the ESU 502 and the third electrode 508 of the second ESI 26-2. A third conductor cable 514 extends between the ESU 502 and a return conductor pad 516 that may be placed in contact with patient tissue 518 to act as a return path for current provided on the third electrode 508. Commonly assigned U.S. Provisional Patent Application, Ser. No. 62/513,287, filed May 31, 2017, entitled Electrosurgical Output Stage with Integrated DC Regulator, describes an ESU 502 in accordance with some embodiments, and is expressly incorporated herein in its entirety by this reference.

The first end effector 454-1 of the first bipolar ESI 26-1 includes an articulated jaw that includes first and second jaw members 520, 522 that articulate relative to one another about a pivot axis 524. At least one of the first and second jaw members 520, 522 is mounted to rotatably pivot about the pivot axis 524 between an open position in which the first and second jaws 520, 522 are spaced apart from each other and a closed position for grasping biological tissue 518 between them. The first and second electrodes 504, 506 are mounted upon the jaw members 520, 522 to electrically contact biological tissue 518 grasped between the first and second jaw members 520, 522. During normal operation, while the jaw members 520, 522 grip tissue between them, the ESU 502 may impart a high frequency electrosurgical signal between the first and second electrodes 504, 506 to cause electrical current to flow through a first tissue portion 518-1 grasped between the jaw members 520, 522 to impart heat to the first tissue portion 518-1 to thereby impart an electrosurgical surgical effect such as desiccation, coagulation, or vaporization, for example.

The second end effector 454-2 of the second monopolar ESI 26-2 includes the single third electrode 508 that may be placed in contact with a patient's biological tissue 518. During normal operation, the ESU 502 may impart a high frequency electrosurgical signal between the third electrode 508 and the return conductor pad 516 to cause electrical current to flow through a second tissue portion 518-2 disposed between the third electrode 508 and the return conductor pad 516 to cause electrical current to flow through the second tissue portion 518-2 to impart to the second tissue portion 518-2 an electrosurgical effect. The return conductor pad 516 may have a surface area that is large enough so that patient tissue in physical contact with the pad has a large enough surface area so that return current to the ESU 502 spreads across a wide enough patient tissue area 518 to limit the current density sufficiently to avoid tissue burns or other trauma due to the return current, for example.

In some embodiments, the bipolar first ESI 26-1 uses lower voltage, and therefore lower energy, than the monopolar second ESI 26-2. Because of the lower energy level, bipolar ESI 26-1 may have a more limited ability to cut and coagulate large bleeding areas, and is more ideally used for those procedures where the first biological tissue portion 26-1 can be easily grabbed on both sides by the jaw members 520, 522 containing the first and second electrodes 504, 506. Thus, in bipolar surgery, the electrosurgical current in the patient is restricted to just the tissue between the jaw electrodes 504, 506, which may provide better control over the area being targeted, and help prevent damage to other sensitive tissues. In some embodiments, a typical bipolar ESI may operate at a voltage in a range of approximately 60 Vp-500 Vp, and a typical monopolar second ESI may operate at a voltage in a range of approximately 300 Vp-3,000 Vp, for example.

The first and second conductor cables 510, 512 span a distance between the ESU 502 and the respective first and second ESIs 26-1, 26-2. The first conductor cable 510 includes a cable outer sheath 510S, which may include insulating material that encloses first and second conductor cords 510-1, 510-2 that extend within it. The second conductor cable 512 includes a cable outer sheath 512S, which may include insulating material that encloses a third conductor cord 512-1 that extends within it. Proximal end portions of the respective first and second conductor cables 510P, 512P and of their respective conductor cords are disposed at the ESU 502. Distal end portions of the respective first and second conductor cables 510D, 512D and of their respective conductor cords are respectively disposed at the first and second ESIs 26-1, 26-2. A proximal end portion of the third (return) cable 514P is disposed at the ESU 502, and a distal end portion of the third cable 514D, secured to the return pad 516, may be disposed at patient's anatomical tissue 518. In some embodiments, the length of the first and second cables 510, 512 may be more than a meter, and therefore, the electrical current path between the proximal end portions 510P, 512P and distal end portions 510D, 512D of the first and second conductor cables 510, 512 may span a distance of more than a meter.

The ESU 502 includes a first transformer circuit 540 to selectably couple an electrosurgical signal between the first and second conductor cords 510-1, 510-2 at the proximal end portion 510P of the of the first conductor cable 510. More particularly, a first pair of transformer switches 542, 544 at the ESU 502 are configured to controllably electrically couple and decouple respective first and second terminals 546, 548 of the first transformer 540 to and from the respective first and second conductor cords 510-1, 510-2 at the proximal end portion 510P of the first cable 510. The ESU 502 includes a second transformer circuit 550 to selectably couple an electrosurgical signal between the third conductor cord 512-1 at the proximal end portion 512P of the second cable 512 and the third (return) conductor cable 514. More specifically, a second pair of transformer switches 552, 554 at the ESU 502 are configured to controllably electrically couple and decouple the first and second terminals 556, 558 of the second transformer 550, respectively, to and from the third conductor cord 512-1 at the proximal end portion 512P of the second cable 512 and the third (return) conductor cable 516.

First and second electrical isolation switches 202, 204 are configured to controllably electrically couple and decouple the distal end portion 510D of the first cable 510 to and from first and second instrument conductors 550-1, 550-2 coupled to the first and second electrodes 504, 506 of the first bipolar ESI 26-1. More particularly, a first instrument conductor 550 includes a proximal portion 550-1P and a distal portion 550-1D, and a second instrument conductor 550-2 includes a proximal portion 550-2P and a distal portion 550-2D. The proximal portion 550-1P of the first instrument conductor 550-1 is electrically coupled to the first conductor cord 510-1, and the distal portion of the 510-1D of the first instrument conductor 550-1 is electrically coupled to the first electrode 504. Similarly, the distal portion 550-2D of the second instrument conductor 550-2 is electrically coupled to the second conductor cord 510-2, and the distal portion of the 510-2D of the second instrument conductor 550-2 is electrically coupled to the second electrode 506. The first electrical isolation switch 202 is operatively disposed to selectably couple and decouple the proximal and distal portions 550-1P, 550-1D of the first instrument conductor 550-1. Similarly, the second electrical isolation switch 204 is operatively disposed to selectably couple and decouple the proximal and distal portions 550-2P, 550-2D of the second instrument conductor 550-2. The first and second electrical isolation switches 202, 204 may be opened to electrically isolate the first and second electrodes 504, 506 from the first and second conductor cords 510-1, 510-2. The first and second electrical isolation switches 202, 204 may be closed to electrically couple the first and second electrodes 504, 506 with the respective first and second conductor cords 510-1, 510-2.

In some embodiments, the first and second electrical isolation switches 202, 204 may be disposed within the end effector actuator mechanism 440, which includes first and second mechanical actuators 445, 447 at a distal end portion of the first ESI 26-1. The distal portions 550-1D, 550-2D of the first and second instrument conductors 550-1, 550-2 extend within a hollow tubular shaft 410 between the isolation switches at the proximal end portion 456 shaft 410 and the first end effector 454-1 at a distal end of the shaft 410. Moreover, mechanical wire cables 560-1, 560-2 extend within the hollow tubular shaft 410 between the first and second mechanical actuators 445, 447 and the end effector 454-1. In some embodiments, mechanical actuators rather than electronic actuators are used within the end effector actuator mechanism 440 so as to reduce potential electrical interference with other medical devices such as a patient's pacemaker, for example.

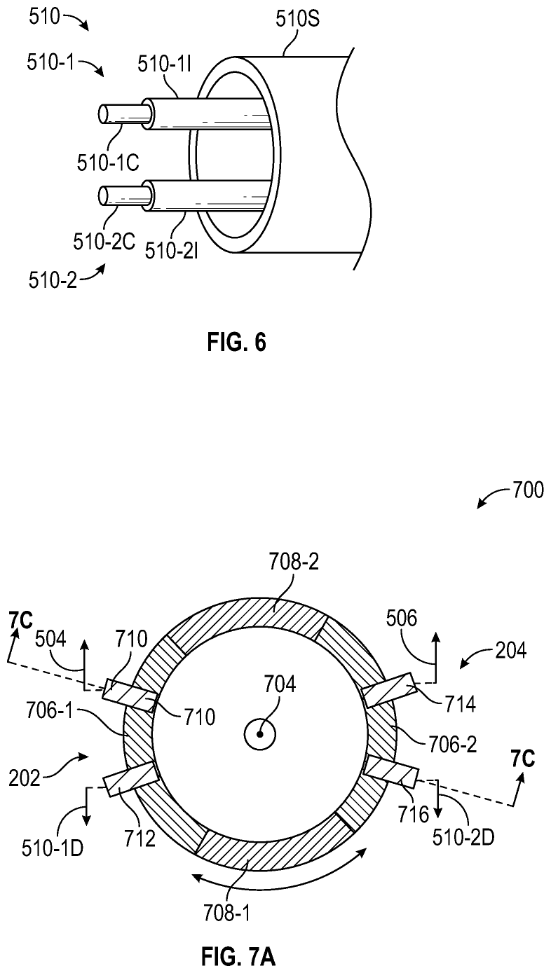

FIG. 6 is an illustrative partially cut-away drawing showing certain details of a portion of the first cable 510. The first cable 510 includes a protective outer sheath 510S, which may be insulating. The first and second conductor cords 510-1, 510-2 are disposed within the protective sheath 510S. The first conductive cord 510-1 includes an insulative outer layer 510-11 and a conductive inner core 510-1C. Similarly, the second conductive cord 510-2 includes an insulative outer layer 510-21 and a conductive inner core 510-2C. Thus, the first isolation switch 202 controllably couples and decouples the distal portion of the conductive inner core 510-1C to and from the first electrode 504 and the second isolation switch 204 controllably couples and decouples the distal portion of the conductive inner core 510-2C to and from the second electrode 506.

Referring again to FIG. 5, during normal operation, during activation of the first bipolar ESI 26-1, the first pair of transformer switches 542, 544 are closed to electrically couple the first and second conductor cords 510-1, 510-2 at the proximal end portion 510P of the first cable 510 to the first transformer 540, and the first and second cable isolation switches 202, 204 are closed to electrically couple the distal end portions of the first and second conductor cords 510-1D, 510-2D to the respective first and second electrodes 504, 506 of the respective first and second jaw members 520, 522. An electrosurgical signal is thereby provided between the first and second electrodes 504, 506. Also during normal operation, during activation of the first bipolar ESI 26-1, the second pair of transformer switches 552, 554 are open to electrically decouple the proximal end portion 512P of the second cable 512 and the third (return) cable 514 from the second transformer 550.

Still referring to FIG. 5, conversely, during normal operation, during activation of the second monopolar ESI 26-2, the second pair of transformer switches 552, 554 are closed to electrically couple the proximal end portion 512P of the second cable 512 and the third (return) conductor cable 514 to the second transformer 550. Also, during activation of the second monopolar ESI 26-2, the first pair of transformer switches 542, 544 are opened to electrically decouple the proximal end portion 510P of the first cable 510 from the first transformer 540, and the first and second cable isolation switches 202, 204 are opened to electrically decouple the distal end portions of the first and second conductor cords 510-1D, 510-2D, at the distal end portion 510D of the first cable 510, from the first and second electrodes 504, 506 of the first end effector 454-1.

The first and second electrical isolation switches 202, 204 help protect a patient from harm. The first and second cable isolation switches 202, 204 are switched to an open state during activation of the second monopolar ESI 26-2, to electrically isolate the proximal portions 550-1P, 550-2P of the first and second instrument conductors 550-1, 550-2 and the respective first and second electrodes 540, 506 electrically coupled thereto, from the first and second conductor cords 510-1, 510-2 to block leakage current from flowing within the first and second electrodes 202, 204. More particularly, without isolation, a leakage current to the first cable 510 may result from capacitive coupling, C_{G}, of the first cable 510 to ground and/or from capacitive coupling, C_{C1/C2}, between the first and second cables 510, 512, for example. That is, during operation of the monopolar ESI 26-2, in the absence of isolation provided by the first and second switches 202, 204, a portion of the electrosurgical current flow between the single third electrode 508 and the return pad 516 of the second monopolar ESI 26-2 may instead leak through patient tissue 518 to the first and/or second electrodes 504, 506 of the first bipolar ESI 26-1 due to such capacitive coupling C_{G} and/or C_{C1/C2}. Such leakage current flow through patient tissue 518 may result in unintended thermal effects such as tissue burn or internal organ damage. Thus, the first and second cable isolation switches 202, 204 help to mitigate or prevent stray leakage current caused by capacitive coupling from flowing through patient tissue 518, which otherwise could result in harm to a patient. Moreover, capacitive coupling such as C_{G} and/or C_{C1/C2} may occur due to a surgical tool (not shown), such as a cautery device or a surgical stapler that contacts a patient's anatomy, and that is energized by lower frequency signals provided on a conductive cable. In other words, a risk of patient harm can arise due to leakage current arising from capacitive coupling even if the second tool is not an electrosurgical instrument energized with a high frequency signal.

### Isolation Switches

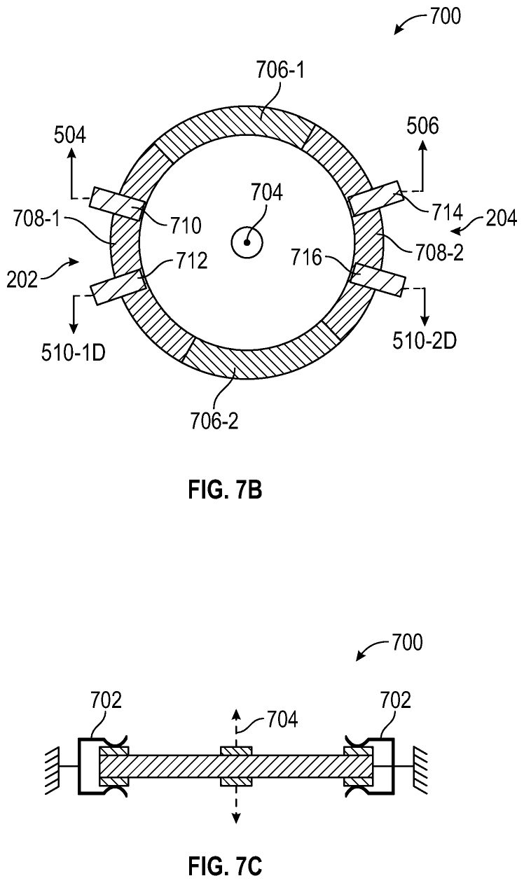

FIGS. 7A-7B are illustrative top elevation views of a mechanical rotary disk switch embodiment 700 of the first and second cable isolation switches 202, 204 of FIGS. 5-6 in closed and open states, respectively, in accordance with some embodiments. FIG. 7C is an illustrative cross-section view along line 7C-7C of FIG. 7A showing double wipe electrical switch contacts 702 in accordance with some embodiments. The rotary disk switch 700 is mounted for rotation about a disk axis 704. The disk switch 700 has alternating top and bottom conductive surface strips 706-1, 706-2 and non-conductive surface strips 708-1, 708-2 spaced evenly about its perimeter. Respective first and second conductive surface strips 706-1, 706-2 are disposed opposite each other at perimeter regions of the disk switch 700 between the first and second non-conductive surface strips 708-1, 708-2. Respective first and second non-conductive surface strips 708-1, 708-2 are disposed opposite each other at perimeter regions of the disk switch 700 between the first and second conductive surface strips 706-1, 706-2.

In some embodiments, the first conductive surface strip 706-1 and the first non-conductive strip 706-1 of rotary disk switch 700 implements the first cable isolation switch 202. The first isolation switch includes first and second terminals 710, 712 disposed at fixed positions to physically contact top and bottom surfaces of the disk switch perimeter. The first terminal 710 is electrically coupled to the first electrode 504 and the second terminal 712 is electrically coupled to the distal end portion of the first conductor cord 510-1D. In some embodiments, the second conductive surface strip 706-2 and the second non-conductive surface strip 708-2 of rotary disk switch 700 implements the second cable isolation switch 204. The first isolation switch includes third and fourth terminals 714, 716 disposed at fixed positions to physically contact top and bottom surfaces of the disk switch perimeter. The third terminal 714 is electrically coupled to the second electrode 506 and the fourth terminal 716 is electrically coupled to the distal end portion of the second conductor cord 510-2D.

The disk switch 700 rotates between a first switch state shown in FIG. 7A and a second switch state shown in FIG. 7B. In the first switch state shown in FIG. 7A, the disk switch 700 is rotated such that top and bottom of the first conductive surface 706-1 electrically contact both the first and second terminals 710, 712 to electrically couple the first electrode 504 with the distal portion of the first conductor cord 510-1D, and the top and bottom of the second conductive surface 706-2 electrically contact both the third and fourth terminals 714, 716 to electrically couple the second electrode 506 with the distal end portion of the second conductor cord 510-2D. In the second switch state shown in FIG. 7B, the disk switch 700 is rotated such that top and bottom of the first non-conductive surface 708-1 electrically contact both the first and second terminals 710, 712 to electrically decouple the first electrode 504 from the distal end portion of the first conductor cord 510-1D, and the top and bottom of the second non-conductive surface 708-2 electrically contact both the third and fourth terminals 714, 716 to electrically decouple the second electrode 506 from the distal end portion of the second conductor cord 510-2D. Thus, in the second switch state, the first and second cable isolation switches 202, 204 are opened to electrically decouple the first cable 510 from the first bipolar ESI 26-1. Moreover, as the disk switch 700 rotates between the first and second switch states, the double wipe electrical contact action of the first through fourth terminals 710-716 helps to ensure good electrical contact by wiping away contaminants such as oxide build-up, for example.

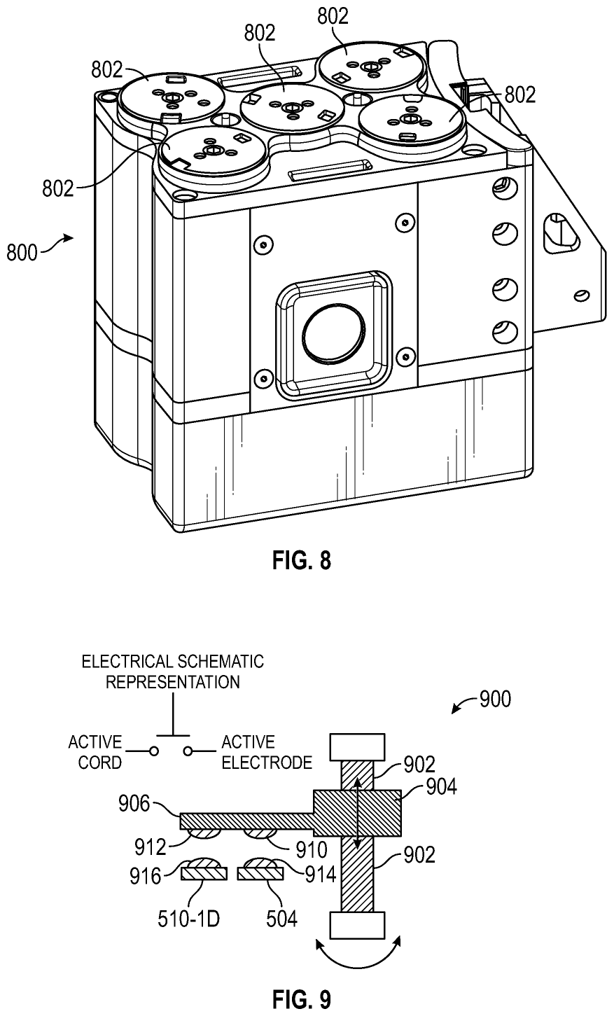

FIG. 8 is an illustrative perspective view of a motor pack 800 to enclose multiple motors (not shown) to controllably rotate individual mechanical rotary drive disks 802 in accordance with some embodiments. One or more of the rotary drive disks 802 may be configured to impart rotational force to actuate the disk switch of FIGS. 7A-7C. The rotary drive disks 802 also may be coupled to impart drive forces to the first and second end effector actuators 445, 447 within the mechanism 440 at the proximal end portion 456 of the shaft 410, to impart forces to wire cables 460-1, 460-2 that extend internally along the length of the shaft 410, to impart motion to an end effector 454 at a distal end portion 450 of the shaft 410. An adapter (not shown) may be disposed between the rotary drive disks 802 of the motor pack 800 and the end effector actuator mechanism 440 to adapt motor-driven rotary disk drive forces to wire cable actuation forces. In some embodiments, one of the rotary disk drives may be configured to selectably rotate the disk switch 700 of FIGS. 7A-7C.

FIG. 9 is an illustrative drawing representing a mechanical lead screw embodiment 900 of a first cable isolation switch 202. A lead screw 902 is threaded through a nut body 904. The nut body 904 is configured such that rotation of the lead screw 902 causes vertical motion of the nut body 904 along the length of the lead screw 902. A cantilever beam 906 formed of an electrically conductive material such as metal, extends from the nut body 904. First and second electrical contacts 910, 912 upstand from the cantilever beam 906. The first electrical contact 910 is vertically aligned with a third electrical contact 914 that is electrically coupled to the first electrode 504. The second electrical contact 912 is vertically aligned with a fourth electrical contact 916 that is electrically coupled to the distal end portion of the first conductor cord 510-1D.

In a first switch state (not shown), the first 910 contact electrically contacts the third contact 914 and the second contact 912 electrically contacts the fourth contact 916 to close the first switch. In a second switch state shown in FIG. 9, the first 910 contact is electrically isolated from the third contact 914 and the second contact 912 is electrically isolated from the fourth contact 916 to open the first switch. It will be appreciated that a similar lead screw arrangement (not shown) may be provided to implement the second cable isolation switch 206.

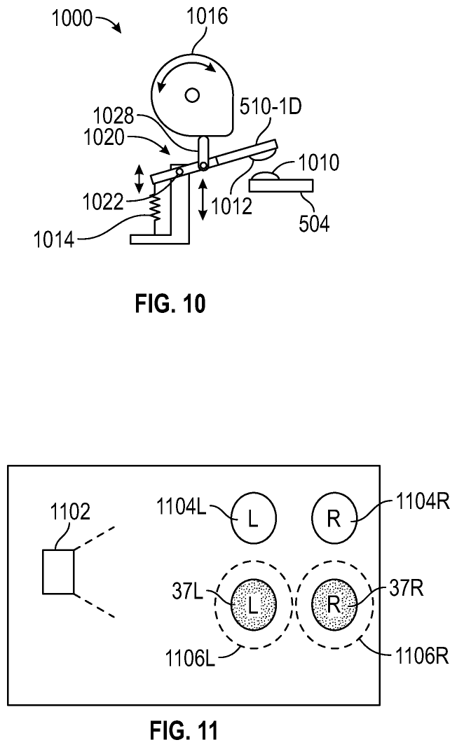

FIG. 10 is an illustrative drawing representing a mechanical cam action switch embodiment 1000 of a first cable isolation switch 202. A first electrical contact 1010 electrically coupled to the first electrode 504 is disposed at a fixed location. A second electrical contact 1012 electrically coupled to the distal end of the first conductor cord 510-1D is disposed at a first end portion of an armature 1020 mounted to a pivot axis 1022. A bias spring 1024 is secured to a second end portion of the armature 1020 to urge the armature 1020 to pivot about the pivot axis 1022 to space apart the first and second electrical contacts 1010, 1012. A rotatable cam 1016 is configured to be rotatable to a first switch state in which a cam surface imparts a force to a cam follower linkage 1028 to the armature 1020 to overcome the spring force and urge the first and second electrical contacts 1010, 1012 into electrical contact to close the first switch 202. The rotatable cam is configured to be rotatable to a second switch state in which a force imparted by cam surface to the cam follower linkage 1028 does not overcome the spring force, which urges the first and second electrical contacts 1010, 1012 apart so that they do not make electrical contact and the first switch 202 is opened. It will be appreciated that a similar cam action arrangement (not shown) may be provided to implement the second cable isolation switch 204.

FIG. 11 is an illustrative drawing representing user interface controls and a sensor device disposed to sense proximity of a user to the respective UI controls. More specifically, FIG. 11 shows left and right foot pedal user interface controls 37L, 37R and a sensor device 902 disposed to sense proximity of a user's left and/or right foot 1104L, 1104R to the respective foot pedals. One or more of the computer processors disposed at the electronics cart 24 is configured to cause the electrical isolation switches 202, 204 to close to electrically couple the first and second electrodes 504, 506 to the first and second conductor cords 510-1, 510-2 in response to the sensor device 1102 sensing a user hovering gesture defined as a user's foot reaching to within a prescribed hovering distance from the foot pedals 1106L, 1104R, indicated by dashed lines 1106L, 1106R. It will that an operator of a surgical instrument may be quite sensitive and the moment when the electrosurgical signal is provided. Mechanical switching may be relatively slow as compared with electronic switching. Commencing closing of the isolation switches 202, 204 in response to a hovering gesture may result in starting the closure of these slower switches sooner so as to reduce the delay perceived by an operator. Although illustrative embodiments have been shown and described, a wide range of modification, change and substitution is contemplated in the foregoing disclosure and in some instances, some features of the embodiments may be employed without a corresponding use of other features. For example, an electrical isolation switch alternatively, may be provided for use with a monopolar electrosurgical instrument. One of ordinary skill in the art would recognize many variations, alternatives, and modifications. Thus, the scope of the disclosure should be limited only by the following claims, and it is appropriate that the claims be construed broadly and in a manner consistent with the scope of the embodiments disclosed herein. The above description is presented to enable any person skilled in the art to create and use a system and method to reduce capacitively coupled leakage current in electrosurgical instruments. Various modifications to the embodiments will be readily apparent to those skilled in the art, and the generic principles defined herein may be applied to other embodiments and applications without departing from the scope of the invention. In the preceding description, numerous details are set forth for the purpose of explanation. However, one of ordinary skill in the art will realize that the invention might be practiced without the use of these specific details. In other instances, well-known processes are shown in block diagram form in order not to obscure the description of the invention with unnecessary detail. Identical reference numerals may be used to represent different views of the same or similar item in different drawings. Thus, the foregoing description and drawings of embodiments in accordance with the present invention are merely illustrative of the principles of the invention. Therefore, it will be understood that various modifications can be made to the embodiments by those skilled in the art without departing from the scope of the invention, which is defined in the appended claims.

1. A surgical instrument comprising:
a shaft that includes an elongated tube that includes a distal end portion and a proximal end portion and that houses an electrical conductor that includes a distal portion and a proximal portion that extends between the proximal end portion and the distal end portion of the elongated tube;
an end effector at the distal end portion of the elongated tube, including an electrode electrically coupled to the distal portion of the electrical conductor; and
a switch at the proximal end portion of the shaft operatively disposed between the distal and proximal portions of the electrical conductor configured to selectably electrically couple and electrically decouple the distal and proximal portions of the electrical conductor.

2. The surgical instrument of claim 1,
wherein the electrode is configured to impart electrical current to the end effector while the switch electrically couples the distal and proximal portions of the electrical conductor and electrical current flows between the electrical conductor and the electrode.

3. The surgical instrument of claim 1,
wherein the switch includes a mechanical actuator at the proximal end portion of the elongated tube.

4. The surgical instrument of claim 3,
wherein the mechanical actuator includes a rotary disk.

5. The surgical instrument of claim 3,
wherein the mechanical actuator includes a lead screw and nut body.

6. The surgical instrument of claim 3,
wherein the mechanical actuator includes a cam and cam follower.

7. The surgical instrument of claim 1,
a mechanical actuator at the proximal end portion of the shaft;
wherein the end effector includes an articulatable member mechanically coupled to the wire cable;
wherein the elongated tube houses a wire cable that extends between the at least one mechanical actuator at the proximal end portion of the elongated tube and the articulatable member at the distal end portion of the elongated tube.

8. The surgical system of claim 1 further including:
wherein the elongated tube houses a wire cable that extends between the proximal end portion and the distal end portion of the elongated tube;
wherein the end effector includes an articulatable member mechanically coupled to the wire cable;
wherein the switch includes a first mechanical actuator at the proximal end portion of the elongated tube; further including:
a second mechanical actuator at the proximal end portion of the elongated tube configured to impart force to the wire cable to impart motion to the articulable member.

9. The surgical system of claim 8,
wherein the articulable member includes a pair of jaws.

10. (canceled)

11. The surgical instrument of claim 1,
wherein the end effector includes a pair of jaws;
wherein the articulatable member includes at least one of the jaw of the pair of jaws;
wherein the electrode is configured to impart electrical current to at least one of the pair of jaws while the switch electrically couples the distal and proximal portions of the electrical conductor and electrical current flows between the electrical conductor and the electrode.

12. A surgical system comprising:
an electrosurgical instrument including a proximal end portion and a distal end portion and including an electrode at the distal end portion;
an electrosurgical signal generator to provide an electrosurgical signal;
a first electrical conductor extending between the electrosurgical instrument and the electrosurgical signal generator;
a second electrical conductor extending between the proximal and distal end portions of the electrosurgical instrument and electrically coupled to the electrode;
a first switch to selectably electrically couple and decouple the electrosurgical signal to and from the second electrical conductor; and
a second switch to selectably electrically couple and decouple the first and second electrical conductors.

13. The surgical system of claim 12,
wherein the second switch includes a mechanical actuator at the proximal end portion of the electrosurgical instrument.

14. The surgical system of claim 13,
wherein the mechanical actuator includes a rotary disk.

15. The surgical system of claim 13,
wherein the mechanical actuator includes a lead screw and nut body.

16. The surgical system of claim 13.
wherein the mechanical actuator includes a cam and cam follower.

17. The surgical system of claim 12 further including:
a processor configured to,
cause the second cause the second switch to electrically couple the first and second electrical conductors while the first switch electrically couples the first electrical conductor to receive the electrosurgical signal, and
cause the second cause the second switch to electrically decouple the first and second electrical conductors while the first switch electrically decouples the first electrical conductor from receiving the electrosurgical signal.

18. The surgical system of claim 12 further including:
a user interface control to receive commands to energize the electrosurgical instrument;
a sensor to sense a hovering gesture by a user at the user interface control;
a processor configured to,
cause the second switch to electrically couple the first and second electrical conductors, in response to sensing a hovering gesture at the user interface control, while the first switch electrically decouples the first electrical conductor from receiving the electrosurgical signal, and

19. The surgical system of claim 12 further including:
a user interface control to receive commands to energize the electrosurgical instrument;
a sensor to sense a hovering gesture by a user at the user interface control;
a processor configured to,
cause the second switch to electrically couple the first and second electrical conductors, in response to sensing a hovering gesture at the user interface control, while the first switch electrically decouples the first electrical conductor from receiving the electrosurgical signal,
cause the second switch to electrically decouple the first and second electrical conductors in response to not sensing a hovering gesture at the user interface control, while the first switch electrically decouples the first electrical conductor from receiving the electrosurgical signal.
cause the second switch to electrically couple the first and second electrical conductors while the first switch electrically couples the first electrical conductor to receive the electrosurgical signal, and

20. A method to provide an electrosurgical signal at a surgical instrument end effector comprising:
in response to a command at a user interface control to energize an electrosurgical instrument,
closing a first switch to electrically provide an electrosurgical signal to a first electrical conductor;
closing a second switch to electrically couple the first electrical conductor to a second electrical conductor electrically coupled to an end effector;
in response to no command at the user interface control to energize the electrosurgical instrument while a hovering gesture is detected at the user interface control,
opening the first switch to not provide the electrosurgical signal to the first electrical conductor;
closing the second switch to electrically couple the first electrical conductor to a second electrical conductor electrically coupled to an end effector;
in response to a command at the user interface control to energize the electrosurgical instrument while no hovering gesture is detected at the user interface control,
opening the first switch to not provide the electrosurgical signal to the first electrical conductor;
opening the second switch to electrically decouple the first electrical conductor from the second electrical conductor electrically coupled to an end effector.
